(19) United States
(12) Patent Application Publication
(10) Pub. No.: US 2007/0123470 A1
(43) Pub. Date: May 31, 2007

(54) Enhancement of growth hormone levels with a dipeptidyl peptidase IV inhibitor and a growth hormone secretagogue

(76) Inventors: Wladimir Hogenhuis, Bridgewater, NJ (US); James H. Mclntyre, Piscataway, NJ (US); Peter S. Kim, Bryn Mawr, NJ (US); Leonardus H.T. Van del Ploeg, Newton, MA (US)

Correspondence Address: MERCK AND CO., INC, P O BOX 2000, RAHWAY, NJ, 07065-0907, US

(21) Appl. No.: 10/575,047

(22) PCT Filed: Oct. 20, 2004
(86) PCT No.: PCT/US04/35112

§ 371 Date: Apr. 4, 2006

Related U.S. Application Data
(60) Provisional application No. 60/514,229, filed on Oct. 24, 2003.

(51) Int. Cl. A61K 38/04; A61K 31/426

(52) U.S. Cl. 514/19; 514/369

(57) ABSTRACT

The present invention is directed to methods for increasing levels of endogenous growth hormone in a mammal by the administration of a combination of a dipeptidyl peptidase IV (DP-IV) inhibitor and a growth hormone secretagogue.

### BACKGROUND OF THE INVENTION

Growth hormone (growth hormone), or somatotropin, is secreted by the pituitary gland, and constitutes a family of hormones for which biological activity is fundamental for the linear growth of a young organism and also for the maintenance of the integrity at its adult state. Growth hormone is known to have the following basic effects on the metabolic processes of the body: increased rate of protein synthesis in all cells of the body; decreased rate of carbohydrate utilization in cells of the body; and increased mobilization of free fatty acids and use of fatty acids for energy.

A deficiency in growth hormone secretion can result in various medical disorders, such as dwarfism. The decrease of growth hormone secretion with age, demonstrated in humans and animals, favors a metabolic shift towards catabolism which initiates or participates to the ageing of an organism. The loss in muscle mass, the accumulation of adipose tissues, the bone demineralization, the loss of tissue regeneration capacity after an injury, which are observed in elderly, correlate with the decrease in the secretion of growth hormone. growth hormone is thus a physiological anabolic agent necessary for the linear growth of children and which controls the protein metabolism in adults.

The pharmacological uses of growth hormone, growth hormone-releasing peptides (GHRP) and growth hormone secretagogues are varied. Treatment with recombinant human growth hormone has been shown to stimulate growth in children with pituitary dwarfism, renal insufficiencies, Turner's syndrome and short stature. A decrease in growth hormone secretion causes changes in body composition during aging. Preliminary studies of one-year treatment with recombinant human growth hormone reported an increase in the muscle mass and in the thickness of skin, a decrease in fat mass with a slight increase in bone density in a population of aged patients. With respect to osteoporosis, recent studies suggest that recombinant human growth hormone does not increase bone mineralization but it is suggested that it may prevent bone demineralization in post-menopausal women. In preclinical and clinical studies, growth hormone has been shown to stimulate protein anabolism and healing in the treatment of burns, AIDS, and cancer, and in wound and bone healing.

Compounds that are inhibitors of the dipeptidyl peptidase IV (“DP-IV” or “DPP-IV”) enzyme are also under investigation as drugs that may be useful in the treatment of diabetes, and particularly type 2 diabetes. The usefulness of DP-IV inhibitors in the treatment of type 2 diabetes is based on the fact that DP-IV in vivo readily inactivates glucagon like peptide-1 (GLP-1) and gastric inhibitory peptide (GIP). GLP-1 and GIP are incretins and are produced when food is consumed. The incretins stimulate production of insulin. Inhibition of DP-IV leads to decreased inactivation of the incretins, and this in turn results in increased effectiveness of the incretins in stimulating production of insulin by the pancreas. DP-IV inhibition therefore results in an increased level of serum insulin. Advantageously, since the incretins are produced by the body only when food is consumed, DP-IV inhibition is not expected to increase the level of insulin at inappropriate times, such as between meals, which can lead to excessively low blood sugar (hypoglycemia). Inhibition of DP-IV is therefore expected to increase insulin without increasing the risk of hypoglycemia, which is a dangerous side effect associated with the use of insulin secretagogues.

### SUMMARY OF THE INVENTION

The present invention is directed to methods for increasing levels of endogenous growth hormone in a mammal in need of elevated levels of growth hormone, by the administration of a combination of a dipeptidyl peptidase IV inhibitor and a growth hormone secretagogue. The combination of these two components results in a greater level of endogenous growth hormone than the administration of an equivalent dose of growth hormone secretagogue alone.

### DETAILED DESCRIPTION OF THE INVENTION

The present invention is directed to methods for increasing levels of endogenous growth hormone in a mammal in need of elevated levels of growth hormone, by administering a combination of a dipeptidyl peptidase IV inhibitor and a growth hormone secretagogue. Administration of these two components results in a greater level of endogenous growth hormone than the administration of an equivalent dose of growth hormone secretagogue alone.

One aspect of the present invention is directed to a method for increasing endogenous growth hormone production by the administration of a combination of a dipeptidyl peptidase IV inhibitor, or a pharmaceutically acceptable salt thereof, and a growth hormone secretagogue, or a pharmaceutically acceptable salt thereof, optionally in combination with a pharmaceutically acceptable carrier.

Another aspect of the present invention is a method for elevating the plasma concentration of growth hormone in a mammal by the administration of a dipeptidyl peptidase IV inhibitor, or a pharmaceutically acceptable salt thereof, and a growth hormone secretagogue, or a pharmaceutically acceptable salt thereof, optionally in combination with a pharmaceutically acceptable carrier.

Another aspect of the present invention is a method for the treatment, control, amelioration, or reduction of risk of a disease or disorder associated with growth hormone deficiency in a mammal by the administration of a a dipeptidyl peptidase IV inhibitor, or a pharmaceutically acceptable salt thereof, and a growth hormone secretagogue, or a pharmaceutically acceptable salt thereof, optionally in combination with a pharmaceutically acceptable carrier. Some of the disorders that can be treated by this invention include growth retardation, metabolic disorders associated with growth hormone deficiency, and the treatment of burns, wounds, trauma, and recovery from surgery.

Dipeptidyl peptidase IV (DP-IV) inhibitors are promising drugs under active investigation for the treatment of diabetes. In accordance with the present invention, the combination of a dipeptidyl peptidase IV inhibitor with a growth hormone secretagogue provides an unexpected enhancement of plasma levels of growth hormone in subjects to which it is administered in pharmaceutically acceptable form, compared to administration of growth hormone secretagogues alone or increased insulin levels alone. The combination treatment of a drug that stimulates insulin production or insulin utilization, and a growth hormone secretagogue, has not been previously reported.

It is known that elevated levels or insulin or insulin utilization stimulate the endogenous production of growth hormone. It has not been reported, however, that administration of a drug that stimulates insulin production or insulin utilization, in combinationn with a growth hormone secretagogue, would have a benefical effect on growth hormone levels in a patient.

Use of the combination of a growth hormone secretagogue and a dipeptidyl peptidase IV inhibitor in accordance with the instant invention, provides substantial benefits relative to the administration of exogenous growth hormone or growth hormone mimetics alone. In particular, the use of growth hormone secretagogues and combinations of the instant invention provides a natural pulsatile level of growth hormone in the body, which enhances the natural patterns of growth hormone levels. In a further embodiment, the growth hormone secretagogues and dipeptidyl peptidase IV inhibitors the instant invention may be orally active, thus providing much more convenient dosing and patient management than intravenous, intraperitonal, or subcutaneous injectable dosage forms.

By the term “growth hormone secretagogue” is meant any exogenously administered compound or agent that directly or indirectly stimulates or increases the endogenous release of growth hormone, growth hormone-releasing hormone or somatostatin in an animal, in particular, a human.

The growth hormone secretagogue may be peptidal or non-peptidal in nature, however, the use of a orally active growth hormone secretagogue is preferred. In addition, it is preferred that the growth hormone secretagogue induce or amplify a pulsatile release of endogenous growth hormone.

A representative class of growth hormone secretagogues is set forth in U.S. Pat. No. 5,206,235 as follows: 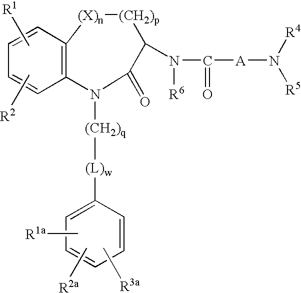
wherein the various substituents are as defined in U.S. Pat. No. 5,206,235.

The most preferred compounds within this class are identified as having the following structures: 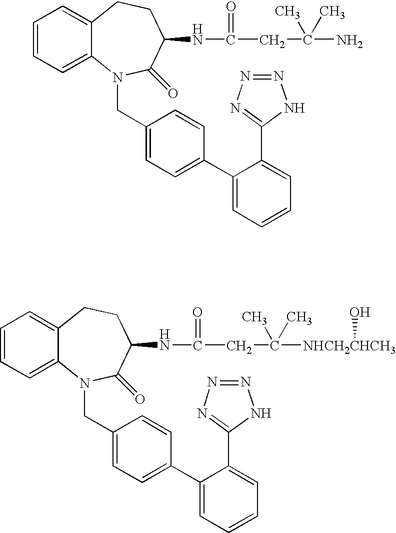
and pharmaceutically acceptable salts thereof.

A representative class of growth hormone secretagogues is set forth in U.S. Pat. No. 5,283,241 and PCT Patent Publication No. 94/05634 as having the following structural formula: 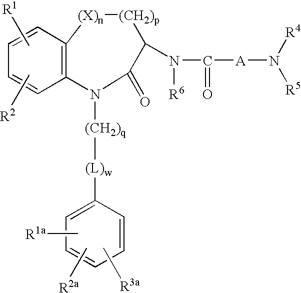
wherein the various substituents are as defined in U.S. Pat. No. 5,283,241 and PCT Patent Publication No. 94/05634.

A representative class of growth hormone secretagogues is disclosed in U.S. Pat. No. 5,536,716 and PCT Patent Pub. No. WO 94/13696 as compounds of the following structural Formulas I and II: 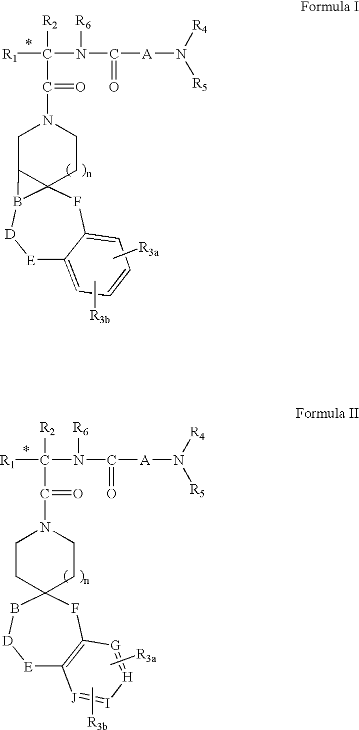
wherein the various substituents are as defined in U.S. Pat. Nos. 5,536,716 and 5,767,124, and PCT Patent Pub. No. WO 94/13696. A specific compound within this class of growth hormone secretagogues which may be employed in the present invention is ibutamoren, N-[1(R)-[(1,2-dihydro-1-methane-sulfonyl-spiro[3H-indole-3,4′-piperdin]-1′-yl)-carbonyl]-2-phenylmethyloxy)ethyl]-2-amino-2-methylpropanamide, having the following structure: 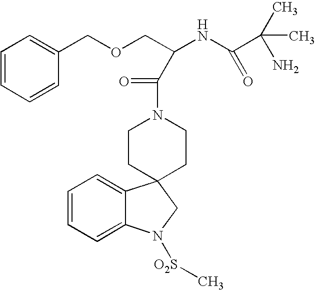
or a pharmaceutically acceptable salt thereof, in particular, the methanesulfonate salt.

A representative class of growth hormone secretagogues is disclosed in U.S. Pat. Nos. 6,107,306, 6,248,717 and 6,596,867. A specific compound within this class of growth hormone secretagogues which may be employed in the present invention is capromorelin, 2-amino-N-{1-(2,4-difluoro-benzyloxymethyl)-2-oxo-2-[3-oxo-3a-pyridin-2-ylmethyl-2-(2,2,2-trifluoro-ethyl)-2,3,3a,4,6,7-hexahydro-pyrazolo[4,3-c]pyridin-5yl]-ethyl}-2-methyl-propionamide, or a pharmaceutically acceptable salt thereof, in particular, the tartrate salt.

By the term dipeptidyl peptidase IV inhibitor (or “DP-IV inhibitor”) is meant any exogenously administered compound or agent that directly or indirectly inhibits or reduces the activity of the enzyme dipeptidyl peptidase type IV.

A class of DP-IV inhibitors is is set forth in and PCT Patent Pub. No. WO 02/076450 and has the following structure: 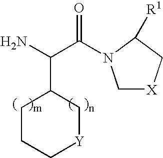
wherein the various substituents are disclosed in WO 02/076540.

Another class of DP-IV inhibitors is disclosed in PCT Patent Pub. No. WO 98/19998 and has the following structure: 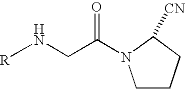
wherein the R group is disclosed in WO 98/19998.

Another class of DP-IV inhibitors is disclosed in U.S. Pat. No. 5,939,560 and has the following structure: 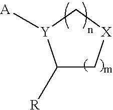
wherein the various substituents are disclosed in U.S. Pat. No. 5,939,560.

Another class of DP-IV inhibitors is disclosed in U.S. Patent Application 2002/0161001 and has the following structure: 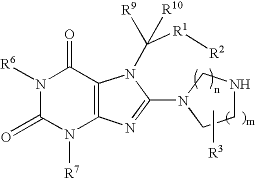
wherein the various substituents are disclosed in US 2002/0161001.

Another class of DP-IV inhibitors is disclosed in U.S. Pat. No. 5,462,928 and has the following structure: 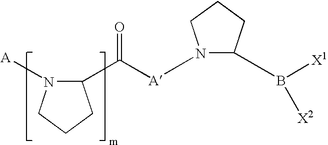
wherein the various substituents are disclosed in U.S. Pat. No. 5,462,928.

Another class of DP-IV inhibitors is disclosed in WO 01/68603 and has the following structure: 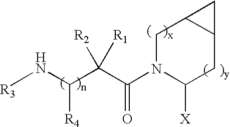
wherein the various substituents are disclosed in WO 01/68603.

Another class of of DP-IV inhibitors is disclosed in WO 02/038541 and has the following structure: 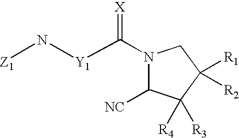
wherein the various substituents are disclosed in WO 02/038541.

Another class of of DP-IV inhibitors is disclosed in WO 02/030891 and has the following structure: 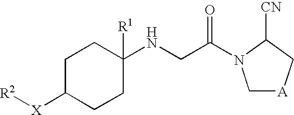
wherein the various substituents are disclosed in WO 02/030891.

Another class of of DP-IV inhibitors is disclosed in WO 02/030890 and has the following structure: 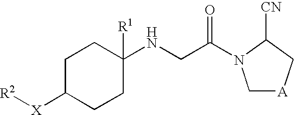
wherein the various substituents are disclosed in WO 02/030890.

Specific examples of DP-IV inhibitors which may be employed in the present invenition include FE-999011, P32/98, DPP728, LAF-237, and SDZ-274444. 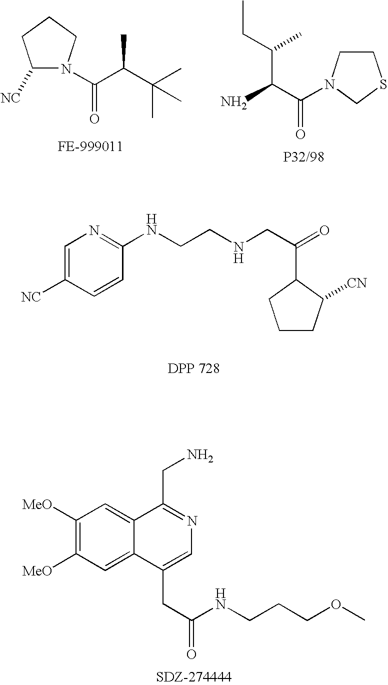
(2S,3S)-2-amino-3-methyl-1-(3,3,4,4-tetrafluoro-pyrrolidin-1-yl)-pentan-1-one;
(2S,3S)-2-amino-1-(3-fluoro-azetidin-1-yl)-3-methyl-pentan-1-one;
(S)-2-amino-2-cyclohexyl-1-(3,3,4,4-tetrafluoro-pyrrolidin-1-yl)-ethanone;
(2S,3R)-2-amino-3-methyl-1-(3,3,4,4-tetrafluoro-pyrrolidin-1-yl)-pentan-1-one;
(S)-2-amino-2-cyclohexyl-1-(3-fluoro-azetidin-1-yl)-ethanone;
(S)-2-amino-3-methyl-1-(3,3,4,4-tetrafluoro-pyrrolidin-1-yl)-butan-1-one;
(S)-2-amino4-methyl-1-(3,3,4,4-tetrafluoro-pyrrolidin-1-yl)-pentan-1-one;
(S)-2-amino-2-cyclohexyl-1-(3,3-difluoro-azetidin-1-yl)-ethanone;
(2S,3S)-2-amino-1-(3,3-difluoro-azetidin-1-yl)-3-methyl-pentan-1-one;
(S)-2-amino-2-cyclohexyl-1-(4,4-difluoro-piperidin-1-yl)-ethanone;
(2S,3S)-2-amino-1-(4,4-difluoro-piperidin-1-yl)-3-methyl-pentan-1-one;
(S)-2-amino-1-(3,3,4,4-tetrafluoro-pyrrolidin-1-yl)-propan-1-one;
(S)-2-amino-1-(3,3-difluoro-azetidin-1-yl)-3-methyl-butan-1-one;
(S)-2-amino-1-(3-fluoro-azetidin-1-yl)-3-methyl-butan-1-one;
(2S, 3R)-2-amino-1-(3-fluoro-azetidin-1-yl)-3-methyl-pentan-1-one;
(2S,3R)-2-amino-1-(3,3-difluoro-azetidin-1-yl)-3-methyl-pentan-1-one;
(S)-2-amino-2-cyclopentyl-1-(3,3-difluoro-azetidin-1-yl)-ethanone;
(S)-2-amino-2-cyclopentyl-1-(3-fluoro-azetidin-1-yl)-ethanone;
(S)-2-amino-2-cyclopentyl-1-(3,3,4,4-tetrafluoro-pyrrolidin-1-yl)-ethanone;
7-[(3R)-3-amino-4-(3,4-difluorophenyl)butanoyl]-2-(trifluoromethyl)-5,6,7,8-tetrahydroimidazo[1,2-α]pyrazine;
7-[(3R)-3-amino-4-(2,5-difluorophenyl)butanoyl]-2-(trifluoromethyl)-5,6,7,8-tetrahydroimidazo[1,2-α]pyrazine;
7-[(3R)-3-amino-4-(2,4,5-difluorophenyl)butanoyl]-2-(trifluoromethyl)-5,6,7,8-tetrahydroimidazo[1,2-α]pyrazine;
7-[(3R)-3-amino-4-(3,4-difluorophenyl)butanoyl]-5,6,7,8-tetrahydroimidazo[1,2-α]pyrazine;
7-[(3R)-3-amino-4-(3,4-difluorophenyl)butanoyl]-3-ethyl-5,6,7,8-tetrahydro-1,2,4-triazolo[4,3-α]pyrazine;
7-[(3R)-3-amino-4-(2,5-difluorophenyl)butanoyl]-3-(trifluoromethyl)-5,6,7,8-tetrahydro-1,2,4-triazolo[4,3-α]pyrazine;
7-[(3R)-3-amino-4-(2,4,5-trifluorophenyl)butanoyl]-3-trifluoromethyl)-5,6,7,8-tetrahydro-1,2,4-triazolo[4,3-α]pyrazine,
(2R)-4-oxo-4-[3-(trifluoromethyl)-5,6-dihydro[1,2,4]triazolo[4,3-a]pyrazin-7(8H)-yl]-1-(2,4,5-trifluorophenyl)butan-2-amine;
1-[2-[(5-cyanopyridin-2-yl)amino]ethylamino]acetylpyrrolidine;
7-[(3R)-3-Amino-4-(3,4-difluorophenyl)butanoyl]-3-methyl-6-(phenylmethyl)-5,6,7,8-tetrahydroimidazo[1,2-a]pyrazine;
7-[(3R)-3-Amino-4-(3,4-difluorophenyl)butanoyl]-3-methyl-6-(phenylmethyl)-5,6,7,8-tetrahydro-1,2,4-triazolo[4,3-a]pyrazine;
7-[(3R)-3-Amino-4-(2,4,5-trifluorophenyl)butanoyl]-8-methyl-3-(trifluoromethyl)-5,6,7,8-tetrahydro-1,2,4-triazolo[4,3-a]pyrazine;
7-[(3R)-3-Amino-4-(2,4,5-trifluorophenyl)butanoyl]-8-methyl-2-(trifluoromethyl)-5,6,7,8-tetrahydroimidazo[1,2-a]pyrazine;
7-[(3R)-3-Amino-4-(3,4-difluorophenyl)butanoyl]-8-(4-fluorophenyl)-2-(trifluoromethyl)-5,6,7,8-tetrahydroimidazo[1,2-a]pyrazine;
7-[(3R)-3-Amino-4-(2,4,5-trifluorophenyl)butanoyl]-8-(methoxycarbonyl)-2-(trifluoromethyl)-5,6,7,8-tetrahydroimidazo[1,2-a]pyrazine;
7-[(3R)-3-Amino-4-(2,4,5-trifluorophenyl)butanoyl]-6-methyl-3-(trifluoromethyl)-5,6,7,8-tetrahydro-1,2,4-triazolo[4,3-a]pyrazine;
7-[(3R)-3-Amino-4-(2,4,5-trifluorophenyl)butanoyl]-6,8-dimethyl-3-(trifluoromethyl)-5,6,7,8-tetrahydro-1,2,4-triazolo[4,3-a]pyrazine;
7-[(3R)-3-Amino-4-(2,5-difluorophenyl)butanoyl]-6-methyl-3-(trifluoromethyl)-5,6,7,8-tetrahydro-1,2,4-triazolo[4,3-a]pyrazine;
(3R)-4-[(3R)-3-Amino-4-(2,4,5-trifluorophenyl)butanoyl]-hexahydro-3-methyl-2H-1,4-diazepin-2-one;
4-[(3R)-3-Amino-4-(2,5-difluorophenyl)butanoyl]hexahydro-1-methyl-2H-1,4-diazepin-2-one;
(3R)-4-[(3R)-3-Amino-4-(2,4,5-trifluorophenyl)butanoyl]-3-benzylhexahydro-1-methyl-2H-1,4-diazepin-2-one;
(3R)-4-[(3R)-3-amino-4-(2,4,5-trifluorophenyl)butanoyl]hexahydro-3-[4-(trifluoromethoxy)benzyl]-2H-1,4-diazepin-2-one;
(3R)-4-[(3R)-3-Amino-4-(2,4,5-trifluorophenyl)butanoyl]-1-tert-butylhexahydro-3-methyl-2H-1,4-diazepin-2-one;
4-[(3R)-3-Amino-4-(2,4,5-trifluorophenyl)butanoyl]hexahydro-5-methyl-2H-1,4-diazepin-2-one;
(3R)-4-[(3R)-3-Amino-4-(2,4,5-trifluorophenyl)butanoyl]hexahydro-3-[(1-oxidopyridin-2-yl)methyl]-2H-1,4-diazepin-2-one;
(3R)-4-[(3R)-3-Amino-4-(2,4,5-trifluorophenyl)butanoyl]hexahydro-3-[(1-oxidopyridin-3-yl)methyl]-2H-1,4-diazepin-2-one;
(3R)-4-[(3R)-3-Amino-4-(2,4,5-trifluorophenyl)butanoyl]hexahydro-3-(1H-pyrazol-1-ylmethyl)-2H-1,4-diazepin-2-one;
(3S)-1-[(2S,3S)-2-Amino-3-(4′-fluoro-1,1′-biphenyl-4-yl)-1-oxobutanyl]-3-fluoropyrrolidine;
(3S)-1-[(2S,3S)-2-Amino-3-[3′-tetrazol-5-yl)-1,1′-biphenyl-yl]-1-oxobutanyl]-3-fluoropyrrolidine;
(3S)-1-[(2S,3S)-2-Amino-3-[3′-(5-oxo-4,5-dihydro-1,2,4-oxadiazol-3-yl)-1,1′-biphenyl-4-yl]-1-oxobutanyl]-3-fluoropyrrolidine;
(3S)-1-[(2S,3S)-2-Amino-3-[4-(6-oxo-1,6-dihydropyridin-3-yl)phenyl]-1-oxobutanyl]-3-fluoropyrrolidine;
(3S)-1-[(2S,3S)-2-Amino-3-[4-(1-methyl-6-oxo-1,6-dihydropyridin-3-yl)phenyl]-1-oxobutanyl]-3-fluoropyrrolidine;
(3S)-1-[(2S,3S)-2-Amino-4-cyclopropyl-3-[4-(1-methyl-6-oxo-1,6-dihydropyridin-3-yl)phenyl]-1-oxobutanyl]-3-fluoropyrrolidine;
(3S)-1-[(2S,3S)-2-Amino-3-[4-(5-bromo-6-oxo-1,6-dihydropyridin-3-yl)phenyl]-1-oxobutanyl]-3-fluoropyrrolidine;
(3S)-1-[(2S,3S)-2-Amino-3-[3′-[(tert-butylamino)carbonyl]-1,1′-biphenyl-4-yl]-1-oxobutanyl]-3-fluoropyrrolidine;
(3S)-1-[(2S,3S)-2-Amino-3-[3′-[[(trifluoromethyl)sulfonyl]amino]-1,1′-biphenyl-4-yl]-1-oxobutanyl]-3-fluoropyrrolidine;
(3S)-1-[(2S,3S)-2-Amino-3-[4-([1,2,4]triazolo[4,3-a]pyridin-6-yl)phenyl]-1-oxobutanyl]-3-fluoropyrrolidine;
(3S)-1-[(2S,3S)-2-Amino-3-carboxy-3-(4′-fluoro-1,1′-biphenyl-4-yl)-1-oxopropanyl]-3-fluoropyrrolidine;
(3S)-1-[(2S,3S)-2-Amino-3-(dimethylaminocarbonyl)-3-(4′-fluoro-1,1′-biphenyl-4-yl)-1-oxopropanyl]-3-fluoropyrrolidine;
1-[(2S,3S)-2-Amino-3-(dimethylaminocarbonyl)-3-(4-[1,2,4)triazolo[1,5-a]pyridin-6-ylphenyl)-1-oxopropanyl]-3,3-difluoropyrrolidine;
1-[(2S,3S)-2-Amino-3-(dimethylaminocarbonyl)-3-(4-[1,2,4]triazolo[1,5-a]pyridin-7-ylphenyl)-1-oxopropanyl]-3,3-difluoropyrrolidine;
1-[(2S,3S)-2-Amino-3-(methylaminocarbonyl)-3-(4-[1,2,4]triazolo[1,5-a]pyridin-6-ylphenyl)-1-oxopropanyl]-3,3-difluoropyrrolidine;
Ethyl 7-[(3R)-3-amino-4-(2,5-difluorophenyl)butanoyl]-5,6,7,8-tetrahydroimidazo[1,2-a]pyrazine-2-carboxylic acid;
7-[(3R)-3-Amino-4-(2,5-difluorophenyl)butanoyl]-5,6,7,8-tetrahydroimidazo[1,2-a]pyrazine-2-carboxylic acid;
7-[(3R)-3-Amino-4-(2,5-difluorophenyl)butanoyl]-N,N-dimethyl-5,6,7,8-tetrahydroimidazo[1,2-a]pyrazine-2-carboxamide;
7-[(3R)-3-Amino-4-(2,5-difluorophenyl)butanoyl]-5,6,7,8-tetrahydroimidazo[1,2-a]pyrazine-2-carboxamide;
7-[(3R)-3-Amino-4-(2,4,5-trifluorophenyl)butanoyl]-2-(trifluoroacetylamino)-5,6,7,8-tetrahydroimidazo[1,2-a]pyrazine;
3-Amino-7-[(3R)-3-amino-4-(2,4,5-difluorophenyl)butanoyl]-2-cyclopropyl-5,6,7,8-tetrahydroimidazo[1,2-a]pyrazine;
7-[(3R)-3-Amino-4-(2,4,5-trifluorophenyl)butanoyl]-2-cyclopropyl-3-(2,2,2-trifluoroacetylamino)-5,6,7,8-tetrahydroimidazo[1,2-a]pyrazine;
Ethyl 7-[(3R)-3-amino-4-(2,4,5-trifluorophenyl)butanoyl]-5,6,7,8-tetrahydro[1,2,4]triazolo[4,3-a]pyrazine-3-carboxylate;
N-(tert-Butyl)-7-[(3R)-3-amino-4-(2,4,5-trifluorophenyl)butanoyl]-5,6,7,8-tetrahydro[1,2,4]triazolo[4,3-a]pyrazine-3-carboxamide;
Ethyl 7-[(3R)-3-amino-4-(2,4,5-trifluorophenyl)butanoyl]-3-chloro-5,6,7,8-tetrahydroimidazo[1,2-a]pyrazine-2-carboxylate;
7-[(3R)-3-Amino-4-(2,4,5-trifluorophenyl)butanoyl]-3-[(R or S)-1-hydroxyethyl]-5,6,7,8-tetrahydroimidazo[1,2-a]pyrazine-2-carboxylic acid;
7-[(3R)-3-Amino-4-(2,4,5-trifluorophenyl)butanoyl]-3-[(S or R)-1-hydroxyethyl]-5,6,7,8-tetrahydroimidazo[1,2-a]pyrazine-2-carboxylic acid;
7-[(3R)-3-Amino-4-(2,4,5-trifluorophenyl)butanoyl]-3-fluoro-2-trifluoromethyl-5,6,7,8-tetrahydroimidazo[1,2-a]pyrazine;
7-[(3R)-3-Amino-4-(2,5-difluorophenyl)butanoyl]-2-(trifluoromethyl)-3-vinyl-5,6,7,8-tetrahydroimidazo[1,2-a]pyrazine;
[7-[(3R)-3-Amino-4-(2,4,5-trifluorophenyl)butanoyl]-5,6,7,8-tetrahydroimidazo[1,2-a]pyrazin-2-yl](cyclopropyl)methanone;
7-[(3R)-3-Amino-4-(2,4,5-trifluorophenyl)butanoyl]-3-methoxy-5,6,7,8-tetrahydro[1,2,4]triazolo[4,3-a]pyrazine;
7-[(3R)-3-Amino-4-(2,4,5-trifluorophenyl)butanoyl]-3-methylthio)-5,6,7,8-tetrahydro[1,2,4]triazolo[4,3-a]pyrazine;
7-[(3R)-3-Amino-4-(2,4,5-trifluorophenyl)butanoyl]-5-methyl-3-(trifluoromethyl)-5,6,7,8-tetrahydro[1,2,4]triazolo[4,3-a]pyrazine;
(5S,8S)- and (5R,8R)-7-[(3R)-3-Amino-4-(2,4,5-trifluorophenyl)butanoyl]-5,8-dimethyl-3-(trifluoromethyl)-5,6,7,8-tetrahydro[1,2,4]triazolo[4,3-a]pyrazine;
(5S,8R)- and (5R,8S)-7-[(3R)-3-Amino-4-(2,4,5-trifluorophenyl)butanoyl]-5,8-dimethyl-3-(trifluoromethyl)-5,6,7,8-tetrahydro[1,2,4]triazolo[4,3-a]pyrazine;
7-[(3R)-3-Amino-4-(2,4,5-trifluorophenyl)butanoyl]-8,8-dimethyl-3-(trifluoromethyl)-5,6,7,8-tetrahydro[1,2,4]triazolo[4,3-a]pyrazine;
7-[(3R)-3-Amino-4-(2,4,5-trifluorophenyl)butanoyl]-5,5-dimethyl-3-(trifluoromethyl)-5,6,7,8-tetrahydro[1,2,4]triazolo[4,3-a]pyrazine;
   or a pharmaceutically acceptable salt thereof.

Representative growth hormone secretagogues of use in the present invention e.g., include the compounds disclosed in the following publications (descriptions of the preparation of such compounds may be found therein): U.S. Pat. Nos. 3,239,345; 4,036,979; 4,411,890; 5,206,235; 5,283,241; 5,284,841; 5,310,737; 5,317,017; 5,374,721; 5,430,144; 5,434,261; 5,438,136; 5,494,919; 5,494,920; 5,492,916; 5,536,716; 5,767,124; 6,107,306; 6,248,717; 6,358,951; 6,429,313; 6,432,945; 6,433,171; 6,482,825; 6,559,150; 6,596,867; 6,603,002; 6,608,028; 6,620,789; 6,632,794; 6,635,619; U.S. Patent Appl'n Nos. US20020042415; US20020045622; US20020049196; US20020137765; US20020165343; EPO Patent Pub. Nos. 0,144,230; 0,513,974; PCT Patent Pub. Nos. WO 89/07110; WO 89/07111; WO 93/04081; WO 94/07486; WO 94/08583; WO 94/11012; WO 94/13696; WO 94/19367; WO 95/03289; WO 95/03290; WO 95/09633; WO 95/11029; WO 95/12598; WO 95/13069; WO 95/14666; WO 95/16675; WO 95/16692; WO 95/17422; WO 95/17423; WO 95/34311; WO 96/02530; WO 96/05195; WO 96/15148; WO 96/22782; WO 96/22997; WO 96/24580; WO 96/24587; WO 96/35713; WO 96/38471; WO 97/00894; WO 97/06803; WO 97/07117; J. Endocrinol Invest., 15(Suppl 4), 45 (1992)); Science, 260, 1640-1643 (Jun. 11, 1993); Ann. Rep. Med. Chem., 28, 177-186 (1993); Bioorg. Med. Chem. Ltrs., 4(22), 2709-2714 (1994); and Proc. Natl. Acad. Sci. USA 92, 7001-7005 (July 1995).

Representative dipeptidyl peptidase IV inhibitors of use in the present invention e.g., include the compounds disclosed in the following publications (descriptions of the preparation of such compounds may be found therein): U.S. Pat. No. 6,124,305 and U.S. Pat. Nos. 5,462,928; 5,939,560; 6,011,155; 6,107,317; 6,110,949; 6,124,305; 6,172,081; 6,380,398; 6,525,083; 6,569,879; 6,699,871; 6,710,040; U.S. Patent Appl'n Nos. 2002/0161001; PCT Patent Pub. Nos. WO 95/15309;WO 97/40832; WO 97/40832; WO 98/18763;WO 98/19998; WO 99/38501; WO 99/46272; WO 99/38501; WO 99/61431; WO 99/67278; WO 99/67279; WO 00/07617; WO 01/60807; WO 01/68603; WO 01/40180; WO 01/81337; WO 01/81304; WO 01/55105; WO 02/02560; WO 02/060388; WO 02/060434; WO 02/064094; WO 02/076450; WO 02/08188; WO 02/14271;WO 02/26729; WO 02/30890;WO 02/30891; WO 02/38541;WO 02/76450; WO 02/83128; WO 03/000180; WO 03/00181; WO 03/00250; WO 03/04496; WO 03/004498; WO 03174500; WO 03/082817; WO 004/007468; WO 04/032836; WO 04/037169; WO 04/043940; WO 04/050022; WO 04/058266; WO 04/064778; WO 04/069162; WO 04/085661; Bioorg. Med. Chem. Lett., 6(10), 1163-1166 (1996); Bioorg. Med. Chem. Lett., 6(22), 2745-2748 (1996), and Expert Opin. Investig. Drugs, 12(1) (2003).

The term “pharmaceutically acceptable salts” refers to salts prepared from pharmaceutically acceptable non-toxic bases or acids including inorganic or organic bases and inorganic or organic acids. Salts derived from inorganic bases include aluminum, ammonium, calcium, copper, ferric, ferrous, lithium, magnesium, manganic salts, manganous, potassium, sodium, zinc, and the like. Particularly preferred are the ammonium, calcium, magnesium, potassium, and sodium salts. Salts in the solid form may exist in more than one crystal structure, and may also be in the form of hydrates. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylene-diamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like.

When the compound of the present invention is basic, salts may be prepared from pharmaceutically acceptable non-toxic acids, including inorganic and organic acids. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid, and the like. Particularly preferred are citric, hydrobromic, hydrochloric, maleic, phosphoric, sulfuric, fumaric, and tartaric acids.

The term “composition” as used herein is intended to encompass a product comprising specified ingredients in predetermined amounts or proportions, as well as any product which results, directly or indirectly, from combination of the specified ingredients in the specified amounts. This term in relation to pharmaceutical compositions is intended to encompass a product comprising one or more active ingredients, and an optional carrier comprising inert ingredients, as well as any product which results, directly or indirectly, from combination, complexation or aggregation of any two or more of the ingredients, or from dissociation of one or more of the ingredients, or from other types of reactions or interactions of one or more of the ingredients. In general, pharmaceutical compositions are prepared by uniformly and intimately bringing the active ingredient into association with a liquid carrier or a finely divided solid carrier or both, and then, if necessary, shaping the product into the desired formulation. In the pharmaceutical composition the active object compound is included in an amount sufficient to produce the desired effect upon the process or condition of diseases. Accordingly, the pharmaceutical compositions of the present invention encompass any composition made by admixing a compound of the present invention and a pharmaceutically acceptable carrier.

Pharmaceutical compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients that are suitable for the manufacture of tablets. The tablets may be uncoated or they may be coated by known techniques to delay disintegration and absorption in the gastrointestinal tract and thereby provide a sustained action over a longer period. Compositions for oral use may also be presented as hard gelatin capsules wherein the active ingredients are mixed with an inert solid diluent, for example, calcium carbonate, calcium phosphate or kaolin, or as soft gelatin capsules wherein the active ingredient is mixed with water or an oil medium, for example peanut oil, liquid paraffin, or olive oil. Aqueous suspensions, oily suspensions, dispersible powders or granules, oil-in-water emulsions, and sterile injectable aqueous or oleagenous suspension may be prepared by standard methods known in the art. By “pharmaceutically acceptable” it is meant the carrier, diluent or excipient must be compatible with the other ingredients of the formulation and not deleterious to the recipient thereof.

The terms “administration of” or “administering a” compound should be understood to mean providing a compound or a prodrug of a compound to the individual in need of treatment in a form that can be introduced into that individuals body in a therapeutically useful form and therapeutically useful amount, including, but not limited to: oral dosage forms, such as tablets, capsules, syrups, suspensions, and the like; injectable dosage forms, such as IV, IM, or IP, and the like; transdermal dosage forms, including creams, jellies, powders, or patches; buccal dosage forms; inhalation powders, sprays, suspensions, and the like; and rectal suppositories.

The term “combination” includes administration of a single dosage formulation which contains a a dipeptidyl peptidase IV inhibitor, or a pharmaceutically acceptable salt thereof, in combination with a growth hormone secretagogue, or a pharmaceutically acceptable salt thereof, as well as administration of each of the two active agents in its own separate dosage formulation. The present invention includes administration of two or more separate dosage formulations at different times, at different dosages and in different frequencies. The separate dosage formulations may be given at different times of the day depending on the duration of action of the individual components. Where separate dosage formulations are used, the individual comp0onents of the composition may be administered at essentially the same time, i.e. concurrently, or at separately staggered times, i.e. sequentially, prior to or subsequent to the administration of the other component. Administration in these various ways is suitable as long as the beneficial pharmaceutical effect of the combination is realized by the patient at substantially the same time. Such beneficial effect is preferably achieved whtn the target blood level concentrations of each active drug are maintained at substantially the same time.

The terms “therapeutically effective amount” means the amount of the subject compound that will elicit the biological or medical response of a tissue, system, animal or human that is being sought by the researcher, veterinarian, medical doctor or other clinician. As used herein, the term “treatment” refers both to the treatment and to the prevention or prophylactic therapy of the mentioned conditions, particularly in a patient who is predisposed to such disease or disorder. The instant combination therefor includes all such regimes of simultaneous or alternating treatment, as well as the use of two dosage formulations that require different routes of administration.

The compositions containing compounds of the present invention may conveniently be presented in unit dosage form and may be prepared by any of the methods well known in the art of pharmacy. The term “unit dosage form” is taken to mean a single dose wherein all active and inactive ingredients are combined in a suitable system, such that the patient or person adminstering the drug to the patient can open a single container or package with the entire dose contained therein, and does not have to mix any components together from two or more containers or packages. Typical examples of unit dosage forms are tablets or capsules for oral administration, single dose vials for injection, or suppositories for rectal administration. This list of unit dosage forms is not intended to be limiting in any way, but merely to represent typical examples in the pharmacy arts of unit dosage forms.

The compositions containing compounds of the present invention may conveniently be presented as a kit, whereby two or more components, which may be active or inactive ingredients, carriers, diluents, and the like, are provided with instructions for preparation of the actual dosage form by the patient or person adminstering the drug to the patient. Such kits may be provided with all necessary materials and ingredients contained therein, or they may contain instructions for using or making materials or components that must be obtained independently by the patient or person administering the drug to the patient.

When treating, preventing, controlling, ameliorating, or reducing the risk of other diseases for which compounds of the present invention are indicated, generally satisfactory results are obtained when the compounds of the present invention are administered at a daily dosage of from about 0.1 milligram to about 100 milligram per kilogram of animal body weight, preferably given as a single daily dose or in divided doses two to six times a day, or in sustained release form. The total daily dosage is from about 1.0 milligrams to about 2000 milligrams, preferably from about 0 1 milligrams to about 20 milligrams per kilogram of body weight. In the case of a 70 kg adult human, the total daily dose will generally be from about 7 milligrams to about 1,400 milligrams. A pharmaceutical composition is preferably provided in a solid dosage formulation comprising about 1 mg, 5 mg, 10 mg, 25 mg, 50 mg, 100 mg, 200 mg or 250 mg active ingredient. This dosage regimen may be adjusted to provide the optimal therapeutic response. The compounds may be administered on a regimen of 1 to 4 times per day, preferably once or twice per day. It will be understood, however, that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

Growth hormone is fundamental for the linear growth of a young organism and also for the maintenance of the integrity at its adult state. Growth hormone is known to have the following basic effects on the metabolic processes of the body: Increased rate of protein synthesis in all cells of the body; Decreased rate of carbohydrate utilization in cells of the body; Increased mobilization of free fatty acids and use of fatty acids for energy.

A deficiency in growth hormone secretion can result in various medical disorders, such as dwarfism. The decrease of growth hormone secretion with age, demonstrated in humans and animals, favors a metabolic shift towards catabolism which initiates or participates to the ageing of an organism. The loss in muscle mass, the accumulation of adipose tissues, the bone demineralization, the loss of tissue regeneration capacity after an injury, which are observed in elderly, correlate with the decrease in the secretion of growth hormone. growth hormone is thus a physiological anabolic agent necessary for the linear growth of children and which controls the protein metabolism in adults.

As is well known to those skilled in the art, the known and potential uses of growth hormone are varied and multitudinous. Thus, the administration of the combinations of this invention for purposes of stimulating the release of endogenous growth hormone can have the same effects or uses as growth hormone itself. These varied uses of the compounds employed in accordance with the present invention may be summarized as follows: stimulating growth hormone release in elderly humans; treating growth hormone deficient adults; prevention of catabolic side effects of glucocorticoids; treatment of osteoporosis; stimulation of the immune system, acceleration of wound healing; accelerating bone fracture repair; treatment of growth retardation; treating acute or chronic renal failure or insufficiency; treatment of physiological short stature, including growth hormone deficient children; treating short stature associated with chronic illness; treating obesity and growth retardation associated with obesity; treating growth retardation associated with Prader-Willi syndrome and Turner's syndrome; accelerating the recovery and reducing hospitalization of burn patients or following major surgery such as gastrointestinal surgery; treatment of intrauterine growth retardation, and skeletal dysplasia; treatment of hypercortisonism and Cushing's syndrome; treatment of peripheral neuropathies; replacement of growth hormone in stressed patients; treatment of osteochondrody-splasias, Noonans syndrome, sleep disorders, schizophrenia, depression, Alzheimer's disease, delayed wound healing, and psychosocial deprivation; treatment of pulmonary dysfunction and ventilator dependency; prevention or treatment of congestive heart failure, improving pulmonary function, restoring systolic and diastolic function, increasing myocardial contractility, decreasing peripheral total vascular resistance, diminishing or preventing loss of body weight and enhancing recovery following congestive heart failure; increasing appetite; attenuation of protein catabolic response after a major operation; treating malabsorption syndromes; reducing cachexia and protein loss due to chronic illness such as cancer or AIDS; accelerating weight gain and protein accretion in patients on TPN (total parenteral nutrition); treatment of hyperinsulinemia including nesidioblastosis; adjuvant treatment for ovulation induction and to prevent and treat gastric and duodenal ulcers; stimulation of thymic development and preventtion of the age-related decline of thymic function; adjunctive therapy for patients on chronic hemodialysis; treatment of immunosuppressed patients and to enhance antibody response following vaccination; increasing the total lymphocyte count of a human, in particular, increasing the T4/T8-cell ratio in a human with a depressed T4/T8-cell ratio resulting, for example, from infection, such as bacterial or viral infection, especially infection with the human immunodeficiency virus; treatment of syndromes manifested by non-restorative sleep and musculoskeletal pain, including fibromyalgia syndrome or chronic fatigue syndrome; improvement in muscle strength, mobility, maintenance of skin thickness, metabolic homeostasis, renal hemeostasis in the frail elderly; stimulation of osteoblasts, bone remodelling, and cartilage growth; prevention and treatment of congestive heart failure; protection of cardiac structure and/or cardiac function; enhancing of recovery of a mammal following congestive heart failure; enhancing and/or improving sleep quality as well as the prevention and treatment of sleep disturbances; enhancing or improving sleep quality by increasing sleep efficiency and augmenting sleep maintenance; prevention and treatment of mood disorders, in particular depression; improving mood and subjective well being in a subject suffering from depression; reducing insulin resistance in humans and animals; stimulation of the immune system in companion animals and treatment of disorders of aging in companion animals; growth promotant in livestock; and stimulation of wool growth in sheep. Further, the instant compounds are useful for increasing feed efficiency, promoting growth, increasing milk production and improving the carcass quality of livestock. In general, the instant compounds are useful in a method of treatment of diseases or conditions which are benefited by the anabolic effects of enhanced growth hormone levels that comprises the administration of an instant compound.

In particular, the instant combinations may be useful in the prevention or treatment of a condition selected from the group consisting of: osteoporosis; catabolic illness; immune deficiency, including that in individuals with a depressed T4/T8 cell ratio; bone fracture, including hip fracture; musculoskeletal impairment in the elderly; growth hormone deficiency in adults or in children; short stature in children; obesity; sleep disorders; cachexia and protein loss due to chronic illness such as AIDS or cancer, and treating patients recovering from major surgery, wounds or burns, in a patient in need thereof. In addition, the instant combinations may be useful in the treatment of illnesses induced or facilitated by corticotropin releasing factor or stress- and anxiety-related disorders, including stress-induced depression and headache, abdominal bowel syndrome, immune suppression, HIV infections, Alzheimer's disease, gastrointestinal disease, anorexia nervosa, hemorrhagic stress, drug and alcohol withdrawal symptoms, drug addiction, and fertility problems.

As a specific embodiment of an oral pharmaceutical composition, a 100 mg potency tablet is composed of 50 mg of a dipeptidyl peptidase IV inhibitor, or a pharmaceutically acceptable salt thereof, and 50 mg of a growth hormone secretagogue, or a pharmaceutically acceptable salt thereof, 268 mg microcrystalline cellulose, 20 mg of croscarmellose sodium, and 4 mg of magnesium stearate. The active, microcrystalline cellulose, and croscarmellose are blended first. The mixture is then lubricated by magnesium stearate and pressed into tablets.

While the invention has been described and illustrated with reference to certain particular embodiments thereof, those skilled in the art will appreciate that various adaptations, changes, modifications, substitutions, deletions, or additions of procedures and protocols may be made without departing from the spirit and scope of the invention. It is intended, therefore, that the invention be defined by the scope of the claims that follow and that such claims be interpreted as broadly as is reasonable.

1-10. (canceled)

11. A method for increasing the level of endogenous growth hormone in a mammal in need thereof, comprising administering a dipeptidyl peptidase IV inhibitor, or a pharmaceutically acceptable salt thereof, and a growth hormone secretagogue, or a pharmaceutically acceptable salt thereof, wherein the level of endogenous growth hormone in the mammal is greater than the level resulting from an equivalent dose of the growth hormone secretagogue alone.

12. The method of claim 11 wherein the mammal is selected from the group consisting of humans, horses, cattle, dogs, cats, rats, mice, pigs, goats, sheep, rabbits and monkeys.

13. The method of claim 12 wherein the mammal is a human.

14. The method of claim 13 wherein the human is an elderly human.

15. The method of claim 13 wherein the dipeptidyl peptidase IV inhibitor is 7-[(3R)-3-amino-4-(2,4,5-trifluorophenyl)butanoyl]-3-(trifluoromethyl)-5,6,7,8-tetrahydro-1,2,4-triazolo[4,3-α]pyrazine, or a pharmaceutically acceptable salt thereof, and the growth hormone secretagogue is ibutamoren, or a pharmaceutically acceptable salt thereof.

16. A method for the treatment, control, amelioration, or reduction of risk of a disease or disorder associated with growth hormone deficiency in a mammal in need thereof by administering a dipeptidyl peptidase IV inhibitor, or a pharmaceutically acceptable salt thereof, and a growth hormone secretagogue, or a pharmaceutically acceptable salt thereof.

17. The method of claim 16 wherein the disease or disorder is growth retardation associated with growth hormone deficiency.

18. The method of claim 17 wherein the disease or disorder is a metabolic disorder associated with growth hormone deficiency.

19. The method of claim 18 wherein disease or disorder is an injury, trauma, burn, a wound in need of healing, or a patient recovering from surgery.

20. The method of claim 16 wherein the dipeptidyl peptidase IV inhibitor is 7-[(3R)-3-amino-4-(2,4,5-trifluorophenyl)butanoyl]-3-(trifluoromethyl)-5,6,7,8-tetrahydro-1,2,4-triazolo[4,3-α]pyrazine, or a pharmaceutically acceptable salt thereof, and the growth hormone secretagogue is ibutamoren, or a pharmaceutically acceptable salt thereof.

21. A pharmaceutical composition comprising a dipeptidyl peptidase IV inhibitor, or a pharmaceutically acceptable salt thereof, and a growth hormone secretagogue, or a pharmaceutically acceptable salt thereof, and a pharmaceutically acceptable carrier.

22. The pharmaceutical composition of claim 21 wherein the dipeptidyl peptidase IV inhibitor is 7-[(3R)-3-amino-4-(2,4,5-trifluorophenyl)butanoyl]-3-(trifluoromethyl)-5,6,7,8-tetrahydro-1,2,4-triazolo[4,3-α]pyrazine, or a pharmaceutically acceptable salt thereof, and the growth hormone secretagogue is ibutamoren, or a pharmaceutically acceptable salt thereof.
